(19) United States
(12) Patent Application Publication
(10) Pub. No.: US 2022/0338830 A1
(43) Pub. Date: Oct. 27, 2022

(54) ADAPTION OF A MEDICAL COMPUTED TOMOGRAPHY IMAGING PROCESS TO AN INDIVIDUAL RESPIRATION BEHAVIOUR OF A PATIENT

(71) Applicant: Siemens Healthcare GmbH, Erlangen (DE)

(72) Inventors: Matthias BAER-BECK, Spardorf (DE); Christian HOFMANN, Erlangen (DE)

(73) Assignee: Siemens Healthcare GmbH, Erlangen (DE)

(21) Appl. No.: 17/724,561

(22) Filed: Apr. 20, 2022

(30) Foreign Application Priority Data
Apr. 23, 2021 (EP) 21170167.7

(51) Int. Cl. A61B 6/00

(52) U.S. Cl. CPC A61B 6/541; A61B 6/5264; A61B 6/5205; A61B 2560/0266

(57) ABSTRACT

A method of adaption of a medical computed tomography imaging process to an individual respiration behaviour of a patient comprises: recording a respiratory movement of a patient by monitoring a respiratory surrogate, and adapting the medical computed tomography imaging process based on the recorded respiratory movement of the patient. An adaption device and a medical computed tomography imaging system are also described.

### CROSS-REFERENCE TO RELATED APPLICATION(S)

The present application claims priority under 35 U.S.C. § 119 to European Patent Application No. EP 21170167.7, filed Apr. 23, 2021, the entire contents of which are incorporated herein by reference.

### FIELD

Embodiments of the present invention relate to a method of adaption of a medical computed tomography (CT) imaging process to an individual respiration behaviour of a patient. Embodiments of the present invention also relate to an adaption device. Further, embodiments of the present invention relate to a medical computed tomography imaging system.

### BACKGROUND

Respiratory information collected by a so-called “respiratory surrogate” is used until now only in dedicated 4D respiratory scan modes and protocols of CT scan systems. Here the respiratory surrogate is used to determine the respiratory phase of a patient either during the scan in order to enable phase correlated scanning or after the scan in order to perform a phase correlated reconstruction of image data based on the scanned raw data.

### SUMMARY

In standard and non-respiratory scans and reconstructions a respiratory surrogate is not recorded at all. The reason for this is that up to now a respiratory surrogate was recorded by an external measurement system, which is quite complicated and complex to attach to the patient. Currently, a new radar-based system for the recording of the respiratory surrogate is being developed. This radar-based system is integrated into a patient table of a CT scan system and is therefore always available without any complicated and time-consuming installation process. This new system for the recording of the respiratory surrogate offers the possibility to make use of the information about the breathing cycle and breathing properties of a patient even in non-respiratory correlated scan processes in order to make patient specific adjustments to the breathing commands before and during the scan.

Breathing commands are contained in almost any CT protocol. Typically, those commands are given right before or during the scan in order to give an advice to the patient how to control his breathing in order to avoid artifacts in the reconstructed images. Currently, the commands are typically pre-recorded speech commands, which can be recorded and stored by the clinical users of the CT scan units. Typically, every clinical imaging task, for example cardiac, dual energy, standard contrast and non-contrast exams have their dedicated commands, which are pre-recorded and stored in specific protocols by the clinical users according to their needs and clinical practice. The drawback of those pre-recorded commands is that they do not take into account the breathing properties of the current patient and that they cannot adapt to the current breathing status of the patient. For example, there is no way to detect whether the patient is following the commands or not, which can in the worst-case yield to non-diagnostic images and the need for a re-scan.

Hence, the inventors have discovered a problem underlying embodiments of the present invention, which is to improve the effectivity of a scan process, which is influenced by a respiratory movement of a patient.

That problem is solved by a method of adaption of a medical computed tomography imaging process to an individual respiration behaviour of a patient according to embodiments of the present invention, by an adaption device according to embodiments of the present invention and by a medical computed tomography imaging system according to embodiments of the present invention.

According to the method of adaption of a medical computed tomography imaging process, for example a medical three-dimensional computed tomography (3D CT) imaging process, to an individual respiration behaviour of a patient, it is proposed to use the monitoring of a respiratory surrogate to optimize the breathing guidance prior and during scanning and to guide the clinical user after the scan about the quality of the scan as long as the patient is still on the table. The method comprises the steps of recording the respiratory movement of a patient by monitoring a respiratory surrogate and adapting the medical computed tomography imaging process based on the recorded respiratory movement of the patient. A surrogate has to be understood as a physiological process, which can be detected using sensor technology and which is correlated to another physiological process to be monitored. Hence, the actual interesting physiological process, for example the movement of an internal organ or the movement i.e., the change of a position of a predetermined internal examination area, can be determined based on the observation of the breathing movement of a patient, by correlating the breathing movement with the movement of the interesting internal portion. In particular, the respiratory surrogate represents the respiratory movement of a patient. The information about the respiratory movement can be used as feedback information for controlling breathing commands, which are harmonized with recording time intervals of the medical computed tomography imaging process such that the imaging is not disturbed by the respiratory movement of the patient. Advantageously, the quality of medical images can be improved and the operating expense and time for wasted scan processes can be reduced. Hence, the medical computed tomography imaging resources can be used more effectively. Further, the comfort for the patient is increased, since the number of additional scan attempts and the time for these attempts can be reduced. Furthermore, the radiation exposure due to additional scan attempts can also be reduced.

The adaption device according to embodiments of the present invention, preferably for a CT system, comprises a monitoring unit for recording the respiratory movement of a patient by monitoring a respiratory surrogate and an adaption unit for adapting the imaging process based on the recorded respiratory movement of the patient. The adaption device shares the advantages of the method according to embodiments of the present invention.

The medical computed tomography imaging system comprises a scan unit for carrying out a medical computed tomography imaging from a patient and an adaption device according to embodiments of the present invention for adapting the medical computed tomography imaging of the scan unit to a recorded respiratory movement of the patient. Further, the medical computed tomography imaging system includes a sensor device, for example a radar sensor, for acquiring sensor data concerning the respiratory movement of the patient. The medical computed tomography imaging system shares the advantages of the method according to embodiments of the present invention.

The essential components of the scan adaption device according to embodiments of the present invention can for the most part be designed in the form of software components and if required, by further adding some hardware as for example a respiratory movement sensor like a radar sensor. This applies in particular to the monitoring unit and the adaption unit of the scan adaption device but also parts of the input interfaces. In principle, however, some of these components can be implemented also in the form of software-supported hardware, for example FPGAs or the like, especially when it comes to particularly fast calculations. Likewise, the required interfaces, for example if it is only a matter of transferring data from other software components, can be designed as software interfaces. However, they can also be designed as hardware-based interfaces that are controlled by suitable software. Furthermore, some parts of the above-mentioned components may be distributed and stored in a local or regional or global network or a combination of a network and software, in particular a cloud system.

A largely software-based implementation has the advantage that medical computed tomography imaging systems that have already been used, can easily be retrofitted by a software update in order to work in the manner according to embodiments of the present invention. In this respect, the object is also achieved by a corresponding computer program product with a computer program that can be loaded directly into a memory device of for example a control device of a medical computed tomography imaging system, with program sections, in order to carry out all steps of the method according to embodiments of the present invention, if the program is executed in the medical computed tomography imaging system, in particular the control device. In addition to the computer program, such a computer program product may contain additional components such as a documentation and/ or additional components, including hardware components such as hardware keys (dongles etc.) for using the software.

For transport to the medical computed tomography imaging system and/ or for storage on or in the medical computed tomography imaging system, a computer-readable medium, for example a memory stick, a hard disk or some other transportable or permanently installed data carrier is used on which the program sections of the computer program that can be read in and executed by a computer unit of the medical computed tomography imaging system are stored. The computer unit can comprise for example, one or more cooperating microprocessors or the like used for this purpose.

The dependent claims and the following description each contain particularly advantageous embodiments and developments of embodiments of the present invention. In particular, the claims of one claim category can also be further developed analogously to the dependent claims of another claim category. In addition, within the scope of the present invention, the various features of different example embodiments and claims can also be combined to form new example embodiments.

In a variant of the method according to embodiments of the present invention the adaption is performed in at least one of the following phases of the medical computed tomography imaging process:
the pre-scan phase,
the scan phase,
the after-scan phase.

Advantageously, the information of the individual breathing behaviour of a patient can be used for the preparation of the scan process, as real time information for a dynamic adaption of the scan process during the actual scan process and as information for the steps after having finished the scan process like the reconstruction of image data or a repetition of the scan in case it has to be expected that the image quality of reconstructed image data would be not sufficient.

In a further variant of the method according to embodiments of the present invention, during the pre-scan phase the adaption comprises the sub-steps of training the patient to breathing optimally for a given scan mode or task by taking into account the patients individual breathing behaviour for the given scan mode or task. Advantageously the recorded breathing behaviour of a patient can be used as feedback information for a feedback loop for training the patient for an individual scan mode or task.

In a further variant of the method according to embodiments of the present invention, during the pre-scan phase the adaption comprises determining patient-dependent, individualized, optimally timed breathing commands. For example, the reaction time of a patient can be determined based on the recorded breathing behaviour of the patient. Hence, the breathing commands can be scheduled such that the determined delay of the reaction of the patient is compensated.

In a further variant of the method according to embodiments of the present invention, during the pre-scan phase a point of time for a breath-holding to start can be determined based on an analysis of a detected pattern of the breathing behaviour of the patient. During the pre-scan phase a breath-holding command may be emitted to the patient to start the breath-holding at the determined time for the breath-holding to start. The breathing command, in particular the breath-holding command, can be emitted to the patient when the time point is well suited for the patients current breathing phase.

In a further variant of the method according to embodiments of the present invention, during the pre-scan phase a point of time for the scan to start can be determined based on the analysis of the detected pattern of the breathing behaviour of the patient. The scan can be delayed, if the patient is currently in an unsuitable breathing phase to follow the given breathing command in time. The scan may be started at the determined point of time for the scan to start. Thus, at the determined point of time for the scan to start, the pre-scan phase ends and the scan phase begins.

The scan may comprise an X-ray emission. The X-ray emission may be, for example, an X-ray emission by an X-ray source of the medical computed tomography imaging system. The X-ray emission may be, for example, an X-ray emission for recording X-ray projections from the patient.

In a further variant of the method according to embodiments of the present invention, during the pre-scan phase a point of time for the X-ray emission to start can be determined based on the analysis of the detected pattern of the breathing behaviour of the patient. The point of time for the breath-holding to start may be earlier and/or not later in time than the point of time for the X-ray emission to start.

The X-ray emission can be delayed, if the patient is currently in an unsuitable breathing phase to follow the given breathing command in time. The X-ray emission may be started at the determined point of the X-ray emission to start.

In particular, the determining of the point of time for the X-ray emission to start can be carried out before the determining of the point of time for the breath-holding to start. The point of time for the breath-holding to start can be determined further based on the determined point of time for the X-ray emission to start.

In a further variant of the method according to embodiments of the present invention, during the pre-scan phase a point of time for the breath-holding to end can be determined based on the analysis of the detected pattern of the breathing behaviour of the patient and/or based on the determined point of time for the breath-holding to start.

The point of time for the breath-holding to end may be that point of time after the determined point of time for the breath-holding to start, at which a continuation of the breath-holding by the patient becomes unlikely, for example, at which a probability for a continuation of the breath-holding by the patient falls below a respective predetermined threshold and/or at which a probability for an abortion of the breath-holding by the patient exceeds a respective predetermined threshold.

A breathing command may be emitted to the patient to stop the breath-holding at the determined time for the breath-holding to end. Thus, unnecessary efforts for breath-holding by the patient can be prevented, increasing the patient's fitness for further examination procedures. A countdown signal indicating the remaining time until the determined time for the breath-holding to end may be emitted to the patient. Thus, the patient's motivation to continue the breath-holding until the remaining time until the determined time for the breath-holding to end can be increased.

In a further variant of the method according to embodiments of the present invention, during the pre-scan phase a point of time for the x-ray emission to end can be determined based on the analysis of the detected pattern of the breathing behaviour of the patient and/or based on the determined point of time for the x-ray emission to start and/or based on the determined point of time for the breath-holding to end. The X-ray emission may be stopped at the determined point of the X-ray emission to end.

The scan phase may end at the determined point of the X-ray emission to end and/or the determined time for the breath-holding to end. The time between the determined point of the X-ray emission to start and the determined point of the X-ray emission to end can be regarded as the scan time.

The determined point of time for the breath-holding to end may be later and/or not earlier in time than the determined point of time for the X-ray emission to end.

In a further variant of the method according to embodiments of the present invention, during the pre-scan phase the scan process is parametrized based on the breathing behaviour and/or based on the determined point of time for the X-ray emission to start and/or based on the determined point of time for the X-ray emission to end.

It can be determined whether the patient is able to hold his breath for performing the scan in the current configuration. If not, the control unit of the medical computed tomography imaging system can give a hint to decrease the scan time, e.g., increase the pitch or switch to another scan mode, e.g., from standard spirals to a high pitch flash scan, if the medical computed tomography imaging system provides that option. In general, these switches can also be done automatically, if local standards and regulations allow for automatic adaptions of the scan parameterization.

In a variant of the method according to embodiments of the present invention, during the scan phase, it is detected, if the patient holds his breath properly, and in case the patient does not hold his breath properly, a command is played to remind and motivate the patient to further hold the breath. Advantageously, feedback of the behaviour of the patient can be used to try to influence the breathing behaviour of the patient in real time to save a current scan operation in case the patient does not exactly follow the breathing commands. Further, the scan process can also be dynamically reparametrized, for example by increasing the pitch, if the patient seems to be not able to hold the breath properly. In that case, in particular if the patient does not improve his breathing behaviour, although he has been admonished to do so, the scan process can be altered such that the speed of recordation of raw data is increased such that the shortened breath-hold time of the patient can be tolerated.

In a further variant of the method according to embodiments of the present invention, during the scan phase, the scan process is aborted in an early stage, if it is detected based on the recorded breathing behaviour that the scan results are likely insufficient and a rescan cannot be avoided. Advantageously, scan resources and time resources can be saved, if it is very likely that a current scan process would lead to a medical image with insufficient image quality.

Further, during the scan phase, it can be detected, if the patient holds his breath properly, and in case the patient does not hold his breath properly, the scan process can be stopped and a pause of the scan process can be carried out at a relevant z-position, where the patient is allowed to stop holding the breath. Under the z-position, it has to be understood the coordinate of the presently recorded slice in z-direction, which is the direction of the system axis of a medical computed tomography imaging system, in particular a CT system. In that case, after the pause, the patient is instructed to perform breath-hold again and the scan process is continued at the z-position, where the scan process was stopped or at a position, where the patient still hold his breath properly. Advantageously, a current scan process can be saved although the patient is transiently not able to follow the breathing commands of the medical computed tomography imaging system properly.

In a further variant of the method according to embodiments of the present invention, in the after-scan phase the individual breathing behaviour of the patient is analysed based on a recorded breathing curve and in case it is detected that the patient did not follow the breathing commands properly during the scan phase, a re-scan is recommended, if severe artifacts are expected. Advantageously, the decision, if the scan has to be repeated or not can be automatically carried out based on the feedback information about the patient's breathing behaviour. In that case, a reconstruction based on deteriorated raw data can be dismissed and hence, time resources and medical examination capacities can be saved.

Furthermore, in a variant of the method according to embodiments of the present invention, in at least one of the pre-scan phase, the scan phase and the after-scan-phase, it is automatically determined based on the recorded breathing behaviour how to adapt the medical computed tomography imaging process. That means that it is decided which measurement or which combination of the above-mentioned measurements for an improvement of the scan process is carried out based on the information of a respiratory surrogate. The analysis for that decision can be implemented using classical signal processing approaches or via deep learning-based algorithms. For example, the analysis of the respiratory curve can be done by some kind of neural network.

In a more detailed described variant of the adaption device, the adaption unit comprises a breathing training unit for emitting breathing commands based on the recorded breathing data and the determined breathing curve. Furthermore, the adaption unit can optionally include a starting point determination unit for setting the starting time point of a scan and a breathing command based on the recorded breathing curve. The adaption unit can also include a parametrization unit for adapting scan parameters and selecting an adapted scan protocol for a recorded individual breathing curve. The parametrization unit may also be able to reparametrize a scan process in real time during a running scan process. Moreover, the adaption unit may include an interrupting unit for stopping the scan process, if it has been detected that the breathing behaviour of the patient deteriorates during the scan process. The interrupting unit stops the scan process by emitting an interruption command to the scan unit of the medical computed tomography imaging system and may for example determine a z-position based on the recorded breathing curve, at which the scan process can be resumed after the patient has recovered. Furthermore, the adaption unit may additionally comprise a recommendation unit for emitting a recommendation of abortion or rescanning in case severe artifacts are to be expected based on the recorded breathing curve. Hence, the adaption device according to that variant may comprise a multiplicity of functionalities for adapting a scan process to an individual breathing behaviour of a patient for improving image quality and for saving scan resources in case it is early detected that a scan process should be aborted due to a lack of quality of the recorded images.

The adaption device according to embodiments of the present invention may be part of a control unit, which is for example part of a medical computed tomography imaging system, preferably a computed tomography system. The control unit can optionally comprise a data storage unit, which stores scan protocols, which can be used for a particular scan mode. Such a scan protocol is transmitted to a driving unit. The driving unit, which may be also part of the control unit, generates control instructions for controlling a scan process. The control instructions are sent to the scan unit of the medical computed tomography imaging system. The control unit may also include a reception unit, which receives raw data from the scan unit during a scan process and which also receives breathing data from a sensor system, for example a radar sensor system, which may be installed in a patient table of the medical computed tomography imaging system, and monitors the breathing movement of the patient. Then, the breathing data are transmitted to an adaption device according to a variant of embodiments of the present invention, which may be also part of the control unit. The adaption device may modify the currently used scan protocol and may then transmit a modified scan protocol to the driving unit. The scan adaption device can also directly transmit instructions to the driving unit, for example a stop instruction, i.e., an interruption command or a start instruction or movement instruction for the patient table or the movable part of the scan unit of the medical computed tomography imaging system. The scan adaption device can also emit a modified breathing command to the driving unit, which is sent to the scan unit and is emitted by an audio system of the scan unit such that the patient can follow the modified breathing command. Further, the scan adaption device may further send a breathing curve to a reconstruction unit, which may be also part of the control unit and reconstructs image data based on the received raw data and the breathing curve. Furthermore, the scan adaption device can emit a recommendation to the user for abortion or rescanning, in case the breathing curve leads to the conclusion that the image quality of the reconstructed medical images would likely be not high enough for a particular application of the medical images generated by the scan process.

### BRIEF DESCRIPTION OF THE DRAWINGS

The present invention is explained below with reference to the figures enclosed once again. The same components are provided with identical reference numbers in the various figures.

The figures are usually not to scale.

FIG. 1 shows a CT-system according to an embodiment of the present invention,

FIG. 2 shows a flow chart diagram illustrating the method of adaption of a medical computed tomography imaging process to an individual respiration behaviour of a patient according to an embodiment of the present invention,

FIG. 3 shows a flow chart diagram illustrating the adaption of the medical computed tomography imaging process in the pre-scan phase,

FIG. 4 shows a flow chart diagram illustrating the adaption of the medical computed tomography imaging process during the scan phase,

FIG. 5 shows a flow chart diagram illustrating the adaption of the medical computed tomography imaging process in the after-scan phase,

FIG. 6 shows a schematic view onto an adaption device according to an embodiment of the present invention,

FIG. 7 shows a schematic view onto a control unit of a CT system, comprising the adaption device shown in FIG. 6.

### DETAILED DESCRIPTION

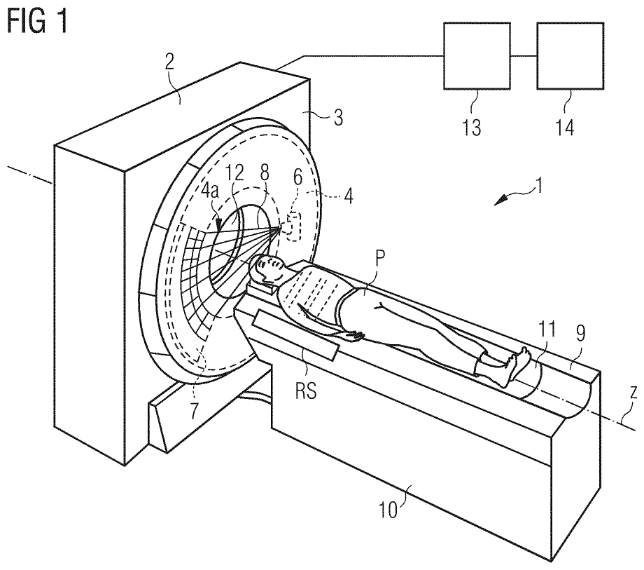

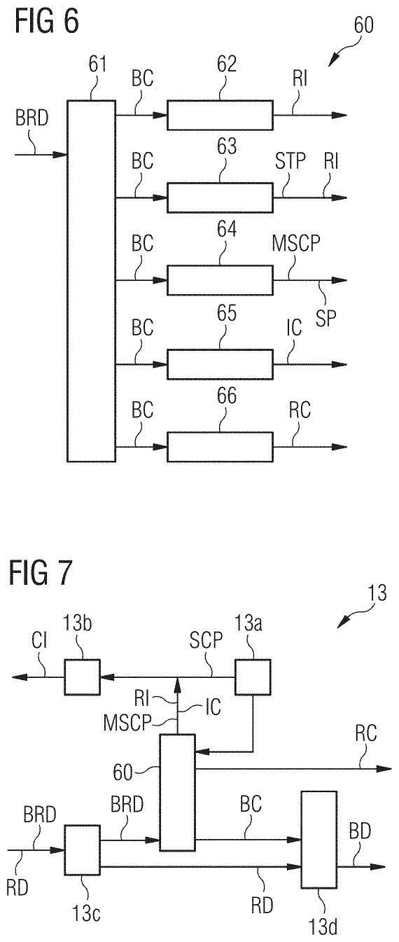

FIG. 1 shows s schematic representation of a computed tomography system 1 comprising an adaption device according to an embodiment of the present invention as later discussed in detail in context with FIG. 6 and FIG. 7 and which may be included by a computer 13 of the computed tomography system 1. The arrangement of the computed tomography system 1 comprises a gantry also called as scan unit 2 with a stationary part 3, also referred to as a gantry frame, and with a part 4, which can be rotated about a system axis z, also referred to as a rotor or drum. The rotating part 4 has an imaging system (X-ray system) 4a, which comprises an X-ray source 6 and an X-ray detector 7, which are arranged on the rotating part 4 opposite one another. The X-ray source 6 and the X-ray detector 7 form the imaging system 4a. When the computed tomography system 1 is in operation, the X-ray source 6 emits X-rays 8 in the direction of the X-ray detector 7, penetrates a measurement object P, for example a patient P, and is transmitted by the X-ray detector 7 in the form of measurement data or measurement signals recorded.

In FIG. 1, a patient table 9 for positioning the patient P can also be seen. The patient table 9 comprises a bed base 10, on which a patient support plate 11, which is provided for actually positioning the patient P, is arranged. The patient support plate 11 can be adjusted relative to the bed base 10 in the direction of the system axis z, i.e. in the z direction, so that it enters an opening 12 such that the patient P can be introduced into the opening 12 of the scan unit 2 for recording X-ray projections from the patient P. A computational processing of the X-ray projections recorded with the imaging system 4a or the reconstruction of sectional images, 3D images or a 3D data set based on the measurement data or measurement signals of the X-ray projections is carried out in an image computer 13 of the computed tomography device 1, wherein the sectional images or 3D images can be displayed on a display device 14. The image computer 13 can also be designed as a control unit for controlling an imaging process and for controlling the scan unit 2 and in particular the imaging system 4a of the scan unit 2. In FIG. 1, also a sensor device, which works as a surrogate monitoring unit RS, is depicted. The surrogate monitoring unit RS emits radar waves (symbolised as dashed lines in FIG. 1) to the area to be scanned, which is the breast of the patient P.

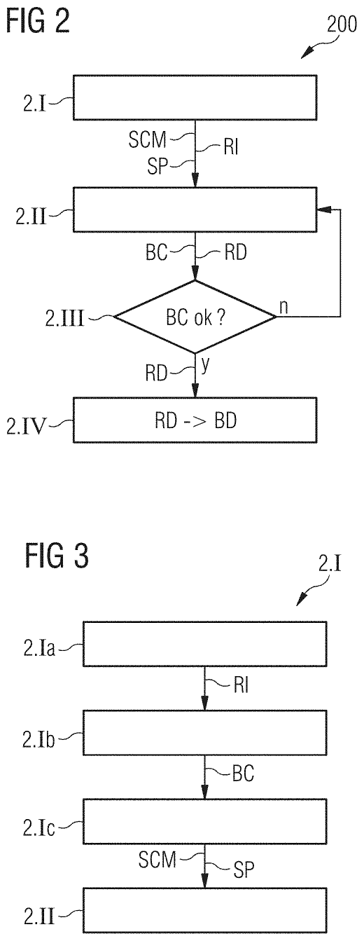

FIG. 2 shows a flow chart diagram 200, which illustrates the method of adaption of a medical computed tomography imaging process to an individual respiration behaviour of a patient P according to an embodiment of the present invention.

In step 2.1 a pre-scan adaption is carried out. Step 2.I comprises a breathing training of a patient P, an adaption of breathing commands RI, a selection of an appropriate scan mode SCM and an adaption of parameters SP of the selected scan mode SCM based on the patient's individual breathing behaviour. Further details of step 2.I are described in context with FIG. 3.

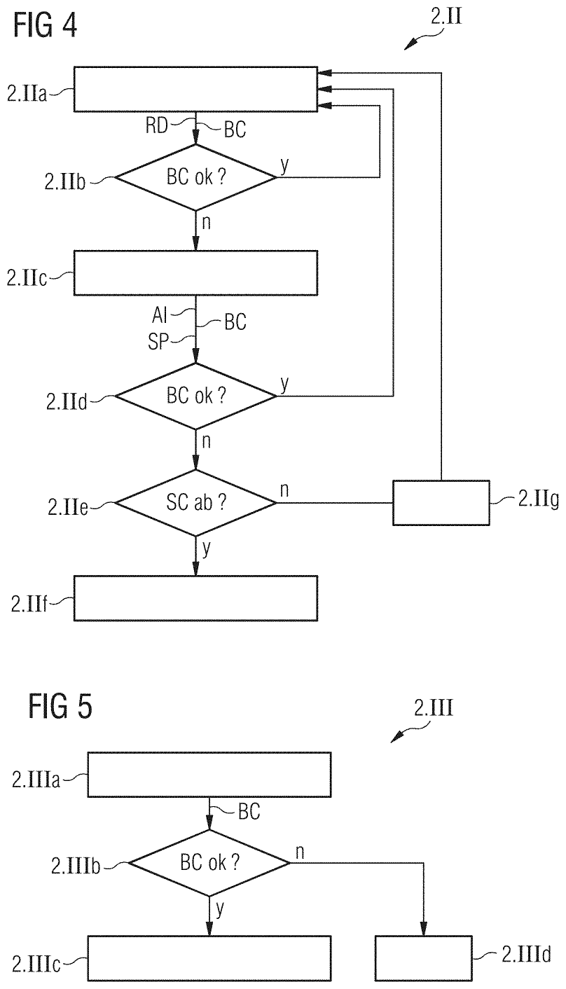

In step 2.II a during-scan adaption is carried out, wherein raw data RD are acquired from the patient. Step 2.II comprises a real time adaption of the scan process during the scanning. As explained in detail in context with FIG. 4, the scan process can be dynamically reparametrized, the scan can be aborted in an early stage or the scan can be interrupted, until the patient P has recovered and then the scan process can be resumed at a z-position, at or after which the breathing curve BC shows an aberration of the breathing behaviour of the patient P from a predetermined breathing curve.

In step 2.III an after-scan adaption of the imaging process is carried out and it is determined based on the recorded breathing curve BC, whether the patient P followed the breathing commands properly during the scan time or not. If that is the case, which is symbolized in FIG. 2 with “y”, step 2.IV can be carried out, wherein a reconstruction based on the recorded raw data RD of the scan is performed and image data BD are reconstructed. If it is determined in step 2.III that the patient did not follow properly enough to the breathing instructions, which is symbolized in FIG. 2 with “n”, it is decided that the image data BD have to be discarded and the scan process has to be repeated and the method goes further with step 2.II. Further details concerning step 2.III are discussed in context with FIG. 5.

In FIG. 3 a flow chart diagram of step 2.I is illustrated. In sub step 2.Ia, a breathing training is performed, wherein a patient P gets some instructions RI for the point of time, when he should hold his breath and for the point of time, when he can proceed with breathing. The training process comprises a feedback loop. That means that in sub step 2.Ib the patient P is monitored and a reaction of the patient P to an instruction RI is recorded. Particularly, a breathing curve BC is recorded. The training can also be used for taking into account the patient's individual breathing behaviour. For example, if the patient P needs some time for reacting to an instruction RI, the individual reaction time of the patient P can be considered for setting the starting time point of a scan process. In sub step 2.Ic based on the recorded breathing curve BC, a scan mode SCM or an adapted set SP of parameters for a scan process is determined. Thereafter, the scan process is carried out with step 2.II, which is described in context with FIG. 4 in detail.

In FIG. 4 a flow chart diagram is illustrated for a detailed description of step 2.II. In sub step 2.IIa, the actual scan process is started and some raw data RD from a scan area of the patient P are recorded. At the same time, a breathing curve BC, describing the breathing behaviour of the patient P, is recorded. At sub step 2.IIb it is determined, if the breathing curve BC is consistent with a predetermined breathing curve. If that is the case, which is represented in FIG. 4 with “y”, the scan process can be continued with sub step 2.IIa. If the breathing curve BC deviates from the predetermined breathing curve, which is represented with “n” in FIG. 4, in sub step 2.IIc a command AI is played out to remind and motivate the patient P to further hold the breath. If it is helpful, the scan process can be dynamically reparametrized, by for example increasing the pitch, if the patient P seems to be not able to hold the breath long enough, or amending some other scan parameters SP. In sub step 2.IId it is determined, if the recorded breathing curve BC is consistent with a possibly amended predetermined breathing curve, which, where appropriate, has been adapted to the amended parameters SP. If that is the case, which is symbolized with “y” in FIG. 4, the scan process proceeds with sub step 2.IIa. If the recorded breathing BC still deviates from the amended breathing curve, which is symbolized with “n” in FIG. 4, the method goes further with sub step 2.IIe. In sub step 2.IIe it is determined, if the scan SC has to be aborted. An abortion can be reasonable, for example in an early stage of a scan process, if it is obvious that the scan result is insufficient and a rescan cannot be avoided. If that is the case, which is symbolized with “y” in FIG. 4, the scan process ends with sub step 2.IIf. In case the scan SC has not been finished, which is symbolized with “n” in FIG. 4, in sub step 2.IIg the scan SC is stopped such that the patient P can recover. After a recovering pause, a z-position is determined, at which the measured breathing curve BC was conform enough with a predetermined breathing curve. Then the scan SC is resumed with sub step 2.IIa at the determined z-position. It is confirmed that most of the sub steps of step 2.II, for example sub step 2.IIe or sub step 2.IIg are optional and can be leaved out in other embodiments of the present invention or arranged in another combination as described in context with FIG. 4.

In FIG. 5 a flow chart is illustrated, which describes step 2.III of the adaption method visualized in FIG. 2 in detail.

In sub step 2.IIIa, after the actual scan process, the recorded breathing curve BC is analysed and compared with a predetermined breathing curve. In sub step 2.IIIb, it is determined, if the recorded breathing curve BC is consistent enough with the predetermined breathing curve. If that is the case, which is symbolised with “y” in FIG. 5, the process ends with sub step 2.IIIc, wherein the reconstruction of recorded raw data RD is carried out. In case the recorded breathing curve BC is not conform enough with the predetermined breathing curve, which is symbolised with “n” in FIG. 5, the scan process ends with sub step 2.IIId, wherein the recorded raw data RD are dismissed and a new scan is recommended.

In FIG. 6 an adaption device 60 is schematically illustrated. The adaption device 60 comprises a monitoring unit 61 for recording breathing data BRD of a respiratory movement of a patient and generating a breathing curve BC based on the recorded breathing data BRD. Further, the adaption device 60 also comprises a breathing training unit 62 for emitting breathing commands RI based on the recorded breathing data BRD and the determined breathing curve BC. Furthermore, the adaption device 60 includes a starting point determination unit 63 for setting the starting time point STP of a scan and a breathing command RI based on the determined breathing curve BC.

The adaption device 60 also includes a parametrization unit 64 for adapting scan parameters SP and selecting an adapted scan protocol MSCP for a determined individual breathing curve BC. The parametrization unit 64 is also able to reparametrize a scan process in real time during a running scan process. Moreover, the adaption device 60 has an interrupting unit 65 for stopping the scan process, if it has been detected that the breathing behaviour of the patient deteriorates during the scan process. The interrupting unit 65 stops the scan process by emitting an interruption command IC to the scan unit 2 and determines a z-position based on the recorded and determined breathing curve BC, at which the scan process can be resumed after the patient P has recovered. Furthermore, the adaption device 60 comprises a recommendation unit 66 for emitting a recommendation RC of abortion or rescanning in case severe artifacts are to be expected based on the breathing curve BC.

In FIG. 7, a control unit 13 is illustrated, as it is for example part of the computed tomography system 1 shown in FIG. 1. The control unit 13 comprises a data storage unit 13a, which stores scan protocols SCP, which can be used for a particular scan mode. Such a scan protocol SCP is transmitted to a driving unit 13b. The driving unit 13b, which is also part of the control unit 13, generates control instructions CI for controlling a scan process. The control instructions CI are sent to the scan unit 2 (shown for example in FIG. 1). The control unit 13 also includes a reception unit 13c, which receives raw data RD from the scan unit 2 during a scan process and which also receives breathing data BRD from a radar sensor system RS (as depicted in FIG. 1), which is installed in the patient table 9 of the computed tomography system 1, and monitors the breathing movement of the patient P. The breathing data BRD are transmitted to an adaption device 60, which is also part of the control unit 13 and is composed of the parts described in context with FIG. 6. The adaption device 60 modifies the currently used scan protocol SCP and transmits a modified scan protocol MSCP to the driving unit 13b. The scan adaption device 60 can also directly transmit instructions to the driving unit 13b, for example a stop instruction, i.e., an interruption command IC or a start instruction or movement instruction for the patient table 9 or the movable part 4a of the scan unit 2. The scan adaption device 60 can also emit a modified breathing command RI to the driving unit 13b, which is sent to the scan unit 2 and emitted by an audio system of the scan unit 2 such that the patient P can follow the modified breathing command RI. Further, the scan adaption device 60 also sends a breathing curve BC to a reconstruction unit 13d, which reconstructs image data BD based on the received raw data RD and the breathing curve BC. Furthermore, the scan adaption device 60 emits a recommendation RC to the user for abortion or rescanning, in case the breathing curve BC leads to the conclusion that the image quality of the reconstructed medical images would likely be not high enough for a particular application of the medical images generated by the scan process.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Further, as noted similarly above, the use of the undefined article “a” or “one” does not exclude that the referred features can also be present several times. Likewise, the term “unit” or “device” does not exclude that it includes several components, which may also be spatially distributed.

The above descriptions are merely embodiments of the present disclosure, but not intended to limit the present disclosure, and any modifications, equivalent replacements, improvements etc. made within the spirit and principle of the present disclosure should be included within the scope of protection of the present disclosure.

What is claimed is:

1. A method of adapting a medical computed tomography imaging process to an individual respiration behaviour of a patient, the method comprising:
recording a respiratory movement of the patient by monitoring a respiratory surrogate; and
adapting the medical computed tomography imaging process based on the respiratory movement of the patient.

2. The method according to claim 1, wherein the adapting is performed in at least one of the following phases of the medical computed tomography imaging process:
a pre-scan phase,
a scan phase, or
a after-scan phase.

3. The method according to claim 2, wherein, during the pre-scan phase the adapting comprises:
training the patient to breath optimally for a given scan mode or task by taking into account an individual breathing behaviour of the patient for the given scan mode or task.

4. The method according to claim 2, wherein, during the pre-scan phase, the method further comprises:
determining a point of time for a breath-holding to start based on an analysis of a detected pattern of a breathing behaviour of the patient.

5. The method according to claim 4, wherein, during the pre-scan phase, the method further comprises:
determining a point of time for an X-ray emission to start based on the analysis of the detected pattern of the breathing behaviour of the patient, the point of time for the breath-holding to start being not later in time than the point of time for the X-ray emission to start.

6. The method according to claim 1, wherein, during a pre-scan phase, the method further comprises:
parameterizing a scan process based on a breathing behaviour of the patient.

7. The method according to claim 2, wherein, during the scan phase, the method further comprises:
determining whether the patient holds their breath properly, and
in case the patient does not hold their breath properly, at least one of
playing out a command to remind and motivate the patient to further hold their breath, or
dynamically reparameterizing a scan process.

8. The method according to claim 2, wherein, during the scan phase, the method further comprises:
aborting a scan process in response to detecting, based on a recorded breathing behaviour of the patient, that scan results are likely insufficient and a rescan cannot be avoided.

9. The method according to claim 2, wherein, during the scan phase, the method further comprises:
determining whether the patient holds their breath properly, and
in case the patient does not hold their breath properly,
stopping a scan process and pausing scanning at a z-position, where the patient is allowed to stop holding their breath,
instructing, after the pausing, the patient to perform a breath-hold again, and
continuing the scan process from the z-position, where the scan process was stopped, or from a z-position, where the patient still holds their breath properly.

10. The method according to claim 2, wherein, during the after-scan phase, the method further comprises:
analyzing an individual breathing behaviour of the patient based on a recorded breathing curve, and
recommending a re-scan in response to detecting that the patient did not follow breathing commands properly during the scan phase and severe artifacts are expected.

11. The method according to claim 2, wherein, in at least one of the pre-scan phase, the scan phase or the after-scan-phase, the method further comprises:
automatically determining, based on a recorded breathing behaviour of the patient, how to adapt the medical computed tomography imaging process to the recorded breathing behaviour of the patient.

12. An adaption device, comprising:
a monitoring unit configured to record a respiratory movement of a patient by monitoring a respiratory surrogate; and
an adaption unit configured to adapt a medical computed tomography imaging process based on the respiratory movement of the patient.

13. A medical computed tomography imaging system, comprising:
a scan unit configured to carry out a medical computed tomography imaging of a patient;
the adaption device according to claim 12, the adaption device configured to adapt the medical computed tomography imaging by the scan unit to the respiratory movement of the patient; and
a sensor device configured to acquire sensor data concerning the respiratory movement of the patient.

14. A non-transitory computer program product with a computer program, which can be loaded directly into a memory device of a medical computed tomography imaging system, the computer program including program sections that, when executed by the medical computed tomography imaging system, cause the medical computed tomography imaging system to perform the method of claim 1.

15. A non-transitory computer readable medium storing program sections that, when executed by a computer unit, cause the computer unit to perform the method of claim 1.

16. The method according to claim 3, wherein, during the pre-scan phase, the method further comprises:
determining a point of time for a breath-holding to start based on an analysis of a detected pattern of a breathing behaviour of the patient.

17. The method according to claim 5, wherein, during the pre-scan phase, the method further comprises:
parameterizing a scan process based on the breathing behaviour of the patient.

18. The method according to claim 5, wherein, during the scan phase, the method further comprises:
determining whether the patient holds their breath properly, and
in case the patient does not hold their breath properly, at least one of
playing out a command to remind and motivate the patient to further hold their breath, or
dynamically reparameterizing a scan process.

19. The method according to claim 5, wherein, during the scan phase, the method further comprises:
aborting a scan process in response to detecting, based on the breathing behaviour of the patient, that scan results are likely insufficient and a rescan cannot be avoided.

20. The method according to claim 5, wherein, during the scan phase, the method further comprises:
determining whether the patient holds their breath properly, and
in case the patient does not hold their breath properly,
stopping a scan process and pausing scanning at a z-position, where the patient is allowed to stop holding their breath,
instructing, after the pausing, the patient to perform a breath-hold again, and
continuing the scan process from the z-position, where the scan process was stopped, or from a z-position, where the patient still holds their breath properly.

21. The method according to claim 5, wherein, during the after-scan phase, the method further comprises:
analyzing the breathing behaviour of the patient based on a recorded breathing curve, and
recommending a re-scan in response to detecting that the patient did not follow breathing commands properly during the scan phase and severe artifacts are expected.

22. The method according to claim 5, wherein, in at least one of the pre-scan phase, the scan phase or the after-scan-phase, the method further comprises:
automatically determining, based on the breathing behaviour of the patient, how to adapt the medical computed tomography imaging process to the breathing behaviour of the patient.

23. The method according to claim 8, wherein the aborting aborts the scan process in an early stage.
